(19) United States
(12) Patent Application Publication
(10) Pub. No.: US 2007/0158189 A1
(43) Pub. Date: Jul. 12, 2007

(54) Disposable biosensor

(75) Inventors: Paul Yang, Taipei (TW); Jen-Chien Chien, Yilan County (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD, CHUNG HO CITY (TW)

(21) Appl. No.: 11/327,360

(22) Filed: Jan. 9, 2006

(51) Int. Cl. G01N 33/487

(52) U.S. Cl. 204/403.02

(57) ABSTRACT

This invention discloses a disposable biosensor and particularly a disposable biosensor concurrently having an electrode strip and a detecting device that detects human biological information such as the level of glucose, total cholesterol, high density lipoprotein, and uric acid by testing human body fluid such as blood, so as to display the test results on a display unit of the biosensor or an external display unit.

### BACKGROUND OF THE INVENTION

1. Field of the Invention

The present invention relates to a disposable biosensor, and more particularly to a biosensor concurrently having an electrode test strip and a detecting device for detecting human physiological information and the contents of biochemical items, so that users can promptly observe the desired detecting results on a display unit of the biosensor or an external display unit.

2. Description of the Related Art

In recent years, some chronic illnesses including diabetes, high blood pressure, hyperglycemia, and gout, etc threaten our health as the economic conditions and level of living standards are improved rapidly. In addition to the diet control and appropriate exercises, people also need to go to a hospital for medical examinations periodically, so that they can know about their current health conditions. Since the concept of household nursing becomes increasingly popular, and there are different kinds of self testing products for a fast, cheap, non-professional operation, and these sensor products including a blood sugar meter, an electronic ear thermometer, an electronic sphygmomanometer, and a cholesterol measuring device, etc waste no time or money to go to a hospital or require no assistance of medial people to carry out the measurements. Users just need to have their own medical measuring instruments to know about their health conditions anytime and anywhere, so as to prevent any possible delay of medical treatments.

Referring to FIG. 1, a general prior art biosensor for detecting human body fluid (such as blood) is a separate electrochemical biosensor comprising a biosensor 100 and a disposable electrode test strip 110 as disclosed in R.O.C. Pat. No. 573735, and the structure of the electrode test strip 110 comprises: an insulating substrate having a dipole electrode system disposed on the same side of the insulating substrate; an insulating layer covered onto the electrode system for defining a reacting area and coupled to an anode connector and a cathode connector of the biosensor; a reacting film disposed in the reacting area, and a plastic net covered onto the reacting area, and finally the plastic net being fixed by a tape with the reacting area exposed from the tape. In the process of testing blood, the aforesaid electrode test strip 110 is inserted, and then the examinee's blood sample is taken and placed onto the enzyme reacting area at the electrode 120 of the electrode test strip 110. The biosensor 100 uses a voltage output device to apply a voltage to the electrode test strip 110 and computes and processes the potential, impedance, or current variation of the electrochemical reaction of the testing object, and then the display device 130 displays the content of biochemical matter such as the level of glucose, total cholesterol, high density lipoprotein, and uric acid, etc.

However, the prior art biosensor 100 is expensive and requires a reacting enzyme on the electrode test strip 110 that usually has an expiration date. As to the users who measure cholesterol, it is a costly burden to buy the biosensors and the electrode test strips since the cholesterol test is usually taken once a month or even longer.

### SUMMARY OF THE INVENTION

It is a primary objective of the present invention to provide a disposable biosensor and particularly a disposable biosensor concurrently having an electrode strip and a detecting device that detects human biological information including the level of glucose, total cholesterol, high density lipoprotein, and uric acid by testing human body fluid such as blood, so that the test results can be displayed on a display unit of the biosensor or an external display unit.

### BRIEF DESCRIPTION OF THE DRAWINGS

FIG. 1 is a schematic view of using a biosensor and an electrode test strip of a prior art;

FIG. 2 is a schematic view of a first preferred embodiment of the present invention;

FIG. 3 is a schematic view of a second preferred embodiment of the present invention;

FIG. 4 is a schematic view of a third preferred embodiment of the present invention; and

FIG. 5 is a schematic view of a further preferred embodiment of the present invention.

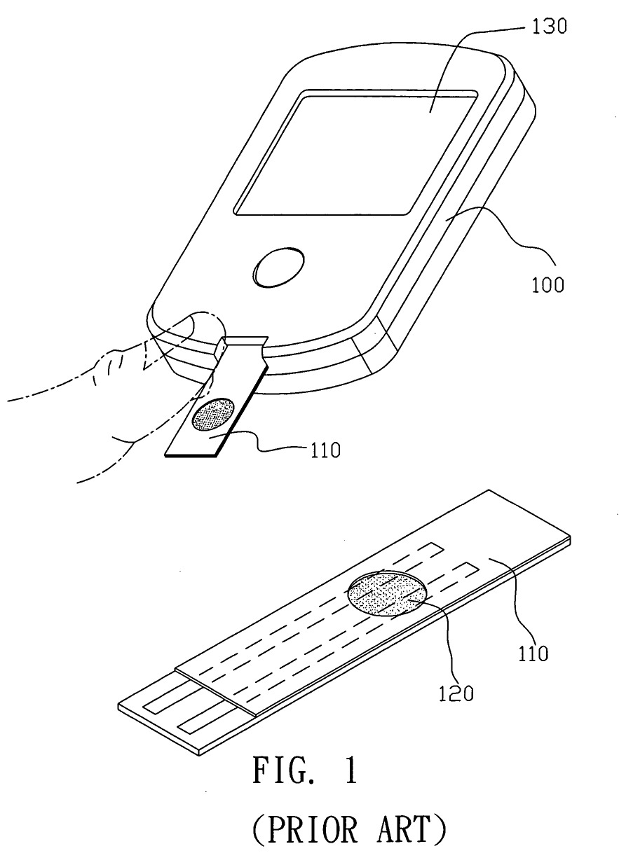

### DETAILED DESCRIPTION OF THE PREFERRED EMBODIMENTS

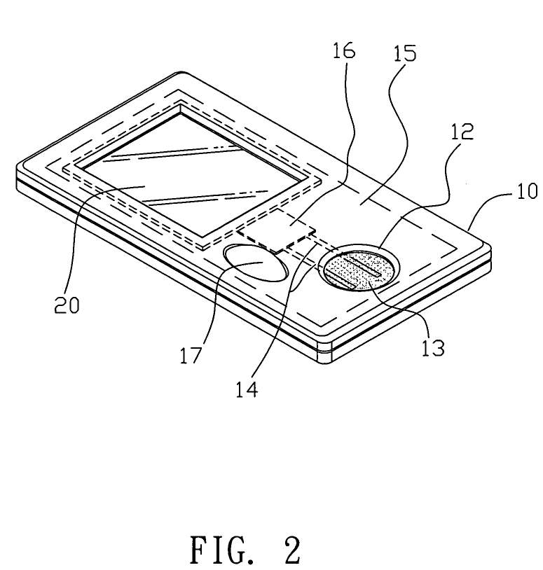

Referring to FIG. 2 for the schematic view of a first preferred embodiment of the present invention, the disposable biosensor of the invention comprises a main body 10; a testing object drop-in opening 12 disposed on a side of the main body 10; an enzyme reacting area 13 disposed under the testing object drop-in opening 12, and one end of the enzyme reacting area 13 is coupled to a working and reference electrode 14 on a circuit board 15, and the working and reference electrode 14 is electrically coupled to a microprocessor 16. The microprocessor 16 is also electrically coupled to a starting switch 17 on the main body 10, such that if a user presses the starting switch 17, an electric signal will be sent to the microprocessor 16, and the microprocessor 16 will compute and process the electric signal sent from the working and reference electrode 14, and the computed digital signal will be displayed on an LCD display device 20.

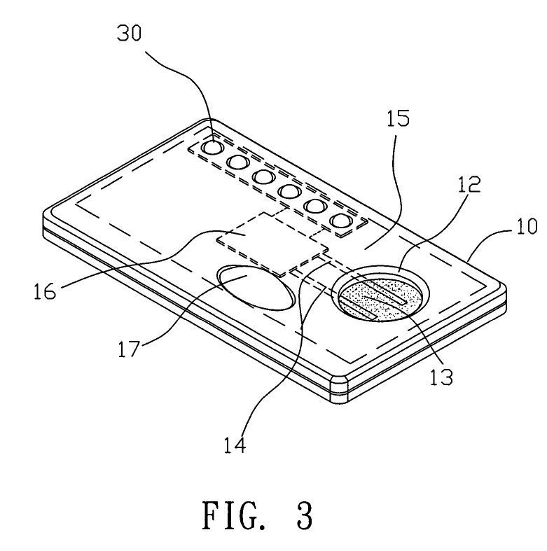

Referring to FIG. 3 for the schematic view of a second preferred embodiment of the present invention, a disposable biosensor of the invention comprises a main body 10; a testing object drop-in opening 12 disposed on a side of the main body 10; an enzyme reacting area 13 disposed under the testing object drop-in opening 12, and an end of the enzyme reacting area 13 is coupled to a working and reference electrode 14 on a circuit board 15, and the working and reference electrode 14 is electrically coupled to a microprocessor 16. The microprocessor 16 is also electrically coupled to a starting switch 17 on the main body 10, such that if a user presses the starting switch 17, a signal will be sent to the microprocessor 16, and the microprocessor 16 will compute and process the signal sent from the working and reference electrode 14, and the computed signal will be displayed on a LED display device 30 in the form of light signals.

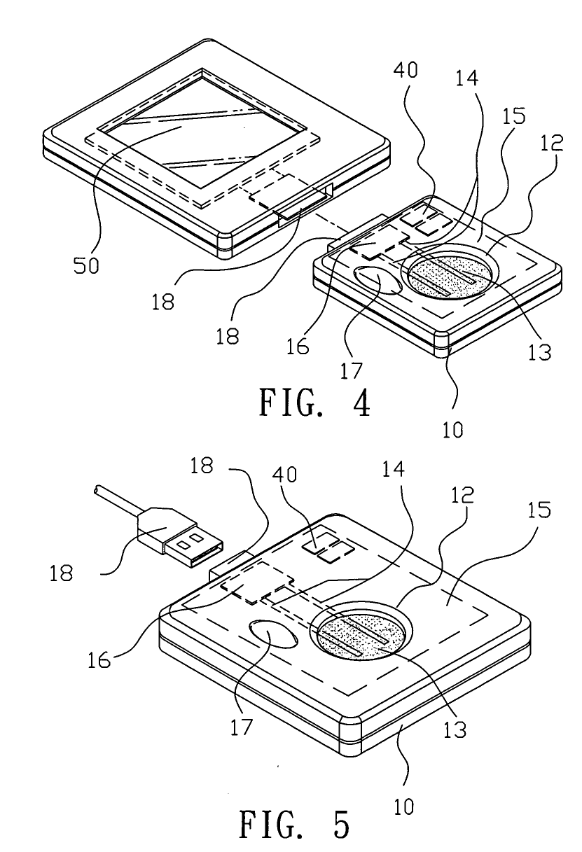

Referring to FIG. 4 for the third preferred embodiment of the present invention, the disposable biosensor of the invention comprises a main body 10; a testing object drop-in opening 12 disposed on a side of the main body 10; an enzyme reacting area 13 disposed under the testing object drop-in opening 12, and an end of the enzyme reacting area 13 is coupled to a working and reference electrode 14 on a circuit board 15, and the working and reference electrode 14 is electrically coupled to a microprocessor 16. The microprocessor 16 is also electrically coupled to a starting switch 17 on the main body 10, such that if a user presses the starting switch 17, a signal will be sent to the microprocessor 16, and the microprocessor 16 will convert the signal sent from the working and reference electrode 14, and the converted signal is stored in a memory 40. A user can use an electric connector (such as a USB or IEEE1394 connector) 18 to connect the main body 10 to an external display device 50. In FIG. 5, the user can connect the main body 10 to an electric connector 18 of an electronic device and display the information through other electronic device such as a computer or a PDA.

In summation of the description above, the present invention directly or indirectly connects the disposable biosensor to a display device and allows users to view the test values immediately and provides a more direct, economic and efficient test.

While the invention has been described by means of specific embodiments, numerous modifications and variations could be made thereto by those skilled in the art without departing from the scope and spirit of the invention set forth in the claims.

What is claimed is:

1. A disposable biosensor for testing the content of biological matters, comprising:
a main body, having a testing object drop-in opening disposed on a side of said main body;
an enzyme reacting area, disposed at the bottom of said testing object drop-in opening, and an end of said enzyme reacting area being coupled to a working and reference electrode of a circuit board;
a microprocessor, electrically coupled to said working and reference electrode;
a starting switch, electrically coupled to said microprocessor, and if said starting switch is pressed, a signal will be sent to said microprocessor, and said microprocessor will process the signal sent from said working and reference electrode; and
a display device, electrically coupled to said microprocessor for displaying a converted digital signal thereon.

2. The disposable biosensor of claim 1, wherein said display device is an LCD display device.

3. The disposable biosensor of claim 1, wherein said display device is a LED display device.

4. A disposable biosensor for testing the content of biological matters, comprising:
a main body, having a testing object drop-in opening disposed on a side of said main body;
an enzyme reacting area, disposed at the bottom of said testing object drop-in opening, and an end of said enzyme reacting area being coupled to a working and reference electrode of a circuit board;
a microprocessor, electrically coupled to said working and reference electrode;
a starting switch, electrically coupled to said microprocessor, and if said starting switch is pressed, a signal will be sent to said microprocessor and said microprocessor will process the signal sent from said working and reference electrode;
a memory, electrically coupled to said microprocessor; and
a display device, detachably and electrically coupled to said memory, for displaying a converted digital signal thereon.

5. The disposable biosensor of claim 4, wherein said display device is an LCD display device.

6. The disposable biosensor of claim 4, wherein said display device is a LED display device.
